(19) United States
(12) Patent Application Publication
(10) Pub. No.: US 2022/0409291 A1
(43) Pub. Date: Dec. 29, 2022

(54) PLANNING AND REAL-TIME UPDATING A 3D TRAJECTORY OF A MEDICAL INSTRUMENT

(71) Applicant: XACT ROBOTICS LTD., Caesarea (IL)

(72) Inventors: Moran SHOCHAT, Zikron Ya'aqov (IL); Ido ROTH, Pardes Hanna (IL); Alon OHEV-ZION, Hadera (IL)

(21) Appl. No.: 17/777,760

(22) PCT Filed: Nov. 26, 2020
(86) PCT No.: PCT/IL2020/051219

§ 371 Date: May 18, 2022

Related U.S. Application Data
(60) Provisional application No. 62/941,586, filed on Nov. 27, 2019.

(51) Int. Cl. A61B 34/20; A61B 34/10

(52) U.S. Cl. CPC A61B 34/20; A61B 34/10; A61B 2034/2051

(57) ABSTRACT

Provided are systems, devices and methods for automated steering of medical instrument in a subject's body for diagnostic and/or therapeutic purposes, wherein the steering of the medical instrument within the subject's body is based on a planned 3D trajectory and real-time updating the 3D trajectory, to allow safely and accurately reaching a target within the subject's body.

### FIELD OF THE INVENTION

The present invention relates to methods, devices and systems for planning and updating in real-time the 3D trajectory of a medical instrument to facilitate the reaching of the medical instrument to a target within the body of a subject, more specifically, the present invention relates to planning and updating in real-time the 3D trajectory of a medical instrument and to steering the medical instrument toward the target according to the planned and/or updated 3D trajectory.

### BACKGROUND

Various diagnostic and therapeutic procedures used in clinical practice involve the insertion of medical tools, such as needles and catheters, percutaneously to a subject's body and in many cases further involve the steering of the medical tools within the body, to reach the target region. The target region can be any internal body region, including, a lesion, tumor, organ or vessel. Examples of procedures requiring insertion and steering of such medical tools include vaccinations, blood/fluid sampling, regional anesthesia, tissue biopsy, catheter insertion, cryogenic ablation, electrolytic ablation, brachytherapy, neurosurgery, deep brain stimulation, various minimally invasive surgeries, and the like.

The guidance and steering of medical tools, such as, needles in soft tissue is a complicated task that requires good three-dimensional coordination, knowledge of the patient's anatomy and a high level of experience. Image-guided automated (e.g., robotic) systems have been proposed for performing these functions.

Some automated insertion systems are based on manipulating robotic arms and some utilize a body-mountable robotic device. These systems include guiding systems that assist the physician in selecting an insertion point and in aligning the medical instrument with the insertion point and with the target, and steering systems that also automatically insert the instrument towards the target.

However, there is still a need in the art for automated insertion and steering devices and systems capable of accurately and reliably determine, update and control, in real-time, 3D trajectory steering of a medical tool within the subject's body to reach the target region, in the most efficient, accurate and safe manner.

### SUMMARY

According to some embodiments, the present disclosure is directed to systems, devices and methods for automated insertion and steering of medical instruments/tools (for example, needles) in a subject's body for diagnostic and/or therapeutic purposes, wherein the steering of the medical instrument within the body of a subject, is based on planning and real-time updating the 3D trajectory of the medical instrument (for example, of the end or tip thereof), within the body of the subject, to allow safely and accurately reaching a target region within the subject's body by the most efficient and safe route. In further embodiments, the systems, devices and methods disclosed herein allow precisely determining and considering the actual location of the tip of the medical instrument within the body to increase effectiveness, safety and accuracy of the medical procedure.

Automatic insertion and steering of medical instruments (such as, needles) within the body, and in particular utilizing real-time trajectory updating, is advantageous over manual steering of such instrument within the body. For example, by utilizing a real-time 3D trajectory updating and steering, the most effective spatio-temporal and safe route of the medical instrument to the target within the body is achieved. Further, the use of real-time 3D trajectory updating and steering increases safety as it reduces the risk of harming non-target regions and tissues within the subjects body, as the 3D trajectory updating may take into account obstacles or any other regions along the route, and moreover, it may take into account changes in the real-time location of such obstacles. Additionally, such automatic steering improves the accuracy of the procedure, which enables reaching small targets and/or targets which are located in areas in the body which are difficult to reach. This can be of particular importance in early detection of malignant neoplasms, for example. In addition, it provides increased safety for the patient, as there is a significant lower risk of human error. Further, according to some embodiments, such a procedure can be executed remotely (e.g., from the adjacent control room or even from outside the medical facility), which is safer for the medical personnel, as it minimizes their radiation exposure during the procedure, as well as their exposure to any infectious diseases the patient may carry, such as COVID-19. Additionally, 3D visualization of the planned and the executed and/or updated trajectory vastly improves the user's ability to supervise and control the medical procedure. Since the automated device can be controlled from a remote site, even from outside of the hospital, there is no longer a need for the physician to be present in the procedure room.

According to some embodiments, there are provided systems for inserting and steering a medical instrument/tool within the body of a subject, utilizing planning and real-time updating the 3D trajectory of the medical instrument within the body of the subject, wherein the system includes an automated insertion and steering device (for example, a robot), a processor and optionally a controller. In some embodiments, the insertion and steering device is configured to insert and steer/navigate a medical instrument in the body of the subject, to reach a target region within the subject's body based on a planned 3D trajectory of the medical instrument, wherein the 3D trajectory is updated in real-time, based on the real-time location of the medical instrument and/or of the target, and wherein the planning and updating of the 3D trajectory is facilitated utilizing the processor, which is further configured to convey real-time steering instructions to the insertion and steering device. According to some exemplary embodiments, the processor may be configured to calculate a pathway (e.g., a 3D trajectory) for the medical instrument from the entry point (also referred to as “insertion point”) to the target, and real-time updating the 3D trajectory, based on the real-time location of the medical instrument and/or the target. In some embodiments, the processor may be further configured to provide instructions, in real-time, to steer (in 3D space) the medical instrument toward the target, according to the planned and/or the updated 3D trajectory. In some embodiments, the steering may be controlled by the processor, via a suitable controller. In some embodiments the steering is controlled in a closed-loop manner, whereby the processor generates motion commands to the steering device via a suitable controller and receives feedback regarding the real-time location of the medical instrument and/or the target, which is then used for real-time updating of the 3D trajectory.

In some embodiments the steering system may be configured to operate in conjunction with an imaging system. In some embodiments, the imaging system may include any type of imaging system (modality), including, but not limited to: X-ray fluoroscopy, CT, cone beam CT, CT fluoroscopy, MRI, ultrasound, or any other suitable imaging modality. In some embodiments, the processor of the system may be further configured to process and show on a display/monitor images, or image-views created from sets of images (or slices), from an imaging system (e.g., CT, MRI), to determine/calculate the optimal 3D trajectory for the medical instrument from an entry point to the target and to update in real-time the 3D trajectory, based on the real-time location of the medical instrument (in particular, the tip thereof) and the target, while avoiding unwanted obstacles and/or reaching desired checkpoints along the route. In some embodiments, the entry point, the target and the obstacles (such as, for example, bones or blood vessels), are manually marked by the physician on one or more of the obtained images or generated image-views.

According to some embodiments, there is provided a method of steering a medical instrument toward a target within a body of a subject, the method includes:
calculating a planned 3D trajectory for the medical instrument from an entry point to a target in the body of the subject;
steering the medical instrument toward the target according to the planned 3D trajectory;
determining if a real-time position of the target deviates from a previous target position;
if it is determined that the real-time position of the target deviates from the previous target position, updating the 3D trajectory of the medical instrument to facilitate the medical instrument reaching the target, and
steering the medical instrument toward the target according to the updated 3D trajectory.

According to some embodiments, the previous target position may be the position of the target as determined or defined prior to the calculating of the planned 3D trajectory. According to some embodiments, the previous target position may be a position of the target as determined or defined during the steering of the medical instrument.

According to some embodiments, updating the 3D trajectory includes calculating a 2D trajectory correction on each of two planes; and superpositioning the two calculated 2D trajectory corrections to form one 3D trajectory correction.

According to some embodiments, the two planes are perpendicular to each other.

According to some embodiments, each of the 2D trajectory corrections may be calculated utilizing an inverse kinematics algorithm.

According to some embodiments, the steering of the medical instrument toward the target within the body may be executed utilizing an automated medical device.

According to some embodiments, the real-time position of the target is determined manually by a user.

According to some embodiments, the real-time position of the target is determined automatically by a processor, using image processing and/or machine learning algorithms.

According to some embodiments, the method may further include real-time tracking the position of the target within the body, to determine the real-time position of the target within the body.

According to some embodiments, the method may further include determining a real- time position of the medical instrument within the body.

According to some embodiments, the real-time position of the medical instrument may be determined manually by a user.

According to some embodiments, the real-time position of the medical instrument may be determined automatically by the processor, using image processing and/or machine learning algorithms.

According to some embodiments, the method may further include real-time tracking the position of the medical instrument within the body to determine the real-time position of the medical instrument within the body.

According to some embodiments, the method may further include determining if the real-time position of the medical instrument within the body deviates from the planned 3D trajectory.

According to some embodiments, determining if the real-time position of the medical instrument within the body deviates from the planned 3D trajectory may be performed continuously.

According to some embodiments, determining if the real-time position of the target deviates from a previous target position may be performed continuously.

According to some embodiments, determining if the real-time position of the medical instrument within the body deviates from the planned 3D trajectory may be performed at checkpoints along the 3D trajectory.

According to some embodiments, determining if the real-time position of the target deviates from a previous target position may be performed at the checkpoints along the 3D trajectory.

According to some embodiments, the checkpoints are predetermined. According to some embodiments, the checkpoints are positioned at a spatial-pattern, a temporal-pattern, or both. According to some embodiments, the checkpoints are spaced along the planned 3D trajectory of the medical instrument. According to some embodiments, the checkpoints are reached at predetermined time intervals.

According to some embodiments, if it is determined that the real-time position of the medical instrument within the body deviates from the planned 3D trajectory, the method further includes adding and/or repositioning one or more checkpoints along the 3D trajectory.

According to some embodiments, adding and/or repositioning the one or more checkpoints along the 3D trajectory may be performed manually by the user. According to some embodiments, adding and/or repositioning the one or more checkpoints along the 3D trajectory may be performed by the processor.

According to some embodiments, calculating the planned 3D trajectory for the medical instrument from the entry point to the target in the body of the subject includes calculating the planned 3D trajectory such that the medical instrument avoids contact with one or more initial obstacles within the body of the subject. According to some embodiments, the method may further include identifying a real-time location of the one or more initial obstacles and/or one or more new obstacles within the body of the subject and wherein updating the 3D trajectory of the medical instrument includes updating the 3D trajectory such that the medical instrument avoids entering the real-time location of the one or more initial obstacles and/or the one or more new obstacles.

According to some embodiments, the method may further include determining one or more secondary target points along the planned 3D trajectory, whereby the medical instrument is to reach the one or more secondary target points along the 3D trajectory, prior to reaching the target.

According to some embodiments, if it is determined that the real-time position of the target deviates from the previous target position, the method may further include determining if the deviation exceeds a predetermined threshold, and whereby the 3D trajectory of the medical instrument is updated only if it is determined that the deviation exceeds the predetermined threshold.

According to some embodiments, the method may further include obtaining one or more images of a region of interest within the body of the subject.

According to some embodiments, the one or more images include images obtained by means of an imaging system, selected from: a CT system, an X-ray fluoroscopy system, an MRI system, an ultrasonic system, a cone-beam CT system, a CT fluoroscopy system, an optical imaging system and an electromagnetic imaging system. According to some embodiments, the one or more images include CT scans.

According to some embodiments, the method may further include displaying the one or more images, or image-views created from the one or more images, on a monitor.

According to some embodiments, determining the real-time position of the medical instrument within the body of the subject includes determining the actual position of a tip of the medical instrument within the body of the subject, and wherein determining the actual position of the tip of the medical instrument within the body of the subject includes:
detecting the medical instrument in one or more images;
defining an end of the detected medical instrument;
determining a compensation value for the end of the medical instrument; and
determining the actual position of the tip of the medical instrument in the body of the subject based on the determined compensation value.

According to some embodiments, the compensation value is selected from a positive compensation value, a negative compensation value and no (zero) compensation.

According to some embodiments, the compensation value may be determined based on a look-up table.

According to some embodiments, the steering of the medical instrument within the body of the subject is performed in a three dimensional space.

According to some embodiments, the method may further include displaying on a monitor at least one of: the planned 3D trajectory, the real-time position of the medical instrument and the updated 3D trajectory.

According to some embodiments, calculating the planned 3D trajectory from the entry point to the target includes:
calculating a 2D trajectory from the entry point to the target on each of two planes; and
superpositioning the two calculated 2D trajectories to form a single 3D trajectory.

According to some embodiments, the two planes are perpendicular to each other.

According to some embodiments, there is provided a system for steering a medical instrument toward a target in a body of a subject, the system includes:
an automated device configured for steering the medical instrument toward the target, the automated device includes one or more actuators and a control head configured for coupling the medical instrument thereto; and
a processor configured for executing the method disclosed herein.

According to some embodiments, the system may further include a controller configured to control the operation of the device.

According to some embodiments, the automated device of the system has at least five degrees of freedom. According to some embodiments, the device has at least one moveable platform. According to some embodiments, the device is configured to be placed on, or in close proximity to, the body of the subject.

According to some embodiments, there is provided a device for steering a medical instrument toward a target in a body of a subject based on a planned and real-time updated 3D trajectory of the medical instrument, the device includes one or more actuators configured for inserting and steering the medical instrument into and within the body of the subject, wherein the updated 3D trajectory is determined by:
real-time tracking of the actual 3D trajectory of the medical instrument within the body;
real-time tracking of the position of the target within the body;
if the real-time 3D trajectory of the medical instrument deviates from the planned 3D trajectory and/or the real-time position of the target deviates from a previous target position, calculating a required 2D trajectory correction on each of two planes and superpositioning the two calculated 2D trajectory corrections to form one 3D trajectory correction.

According to some embodiments, the device may further include a processor configured to calculate the planned and updated 3D trajectory.

According to some embodiments, the device has at least five degrees of freedom. According to some embodiments, the device is an automated device. According to some embodiments, the device is configured to be placed on the body of the subject.

According to some embodiments, there is provided a system for steering a medical instrument into an internal target within a body of a subject, the system includes:
an automated device configured to execute steering of the medical instrument toward the target within the body of the subject;
at least one processor configured to:
   calculate a planned 3D trajectory for the medical instrument from an entry point to a target in the body of the subject;
   generate commands to steer the medical instrument toward the target according to the planned 3D trajectory;
   determine if a real-time position of the target deviates from a previous target position;
   update in real-time the 3D trajectory of the medical instrument; and
   generate commands to steer the medical instrument toward the target according to the updated 3D trajectory;
   and;
at least one controller configured to control the operation of the device based on commands generated by the processor.

According to some embodiments, calculating the planned 3D trajectory from the entry point to the target includes:

calculating a 2D trajectory from the entry point to the target on each of two planes; and superpositioning the two calculated 2D trajectories to form a single 3D trajectory.

According to some embodiments, the two planes are perpendicular to each other. According to some embodiments, each of the 2D trajectories may be calculated utilizing an inverse kinematics algorithm.

According to some embodiments, the at least one processor may be configured to determine if the deviation of the real-time position of the target from the previous target position exceeds a set threshold, and whereby the 3D trajectory of the medical instrument is updated only if it is determined that the deviation exceeds the set threshold.

According to some embodiments, updating the 3D trajectory includes: calculating a 2D trajectory correction on each of two planes; and superpositioning the two calculated 2D trajectory corrections to form one 3D trajectory correction. In some embodiments, the two planes are perpendicular to each other. In some embodiments, each of the 2D trajectory corrections is calculated utilizing an inverse kinematics algorithm.

According to some embodiments, the at least one processor is configured to determine if a real-time position of the medical instrument within the body deviates from the planned 3D trajectory. According to some embodiments, the at least one processor is configured to determine the real-time position of the medical instrument within the body using image processing and/or machine learning algorithms. According to some embodiments, the at least one processor is configured to track, in real-time, the position of the medical instrument within the body, to determine the real-time position of the medical instrument within the body. According to some embodiments, the real-time position of the medical instrument is determined manually by a user.

According to some embodiments, the at least one processor is configured to determine the real-time position of the target using image processing and/or machine learning algorithms. According to some embodiments, the at least one processor is configured to track, in real-time, the position of the target within the body, to determine the real-time position of the target within the body. According to some embodiments, the real-time position of the target is determined manually by a user.

According to some embodiments, determining if the real-time position of the medical instrument within the body deviates from the planned 3D trajectory is performed continuously.

According to some embodiments, determining if the real-time position of the medical instrument within the body deviates from the planned 3D trajectory is performed at checkpoints along the 3D trajectory.

According to some embodiments, determining the real-time position of the medical instrument within the body of the subject includes determining the actual position of a tip of the medical instrument within the body of the subject.

According to some embodiments, determining if the real-time position of the target deviates from a previous target position is performed continuously.

According to some embodiments, determining if the real-time position of the target deviates from a previous target position is performed at the checkpoints along the 3D trajectory.

According to some embodiments, the system may further include or is configured to operate in conjunction with an imaging device.

According to some embodiments, the imaging device may be selected from: a CT device, an X-ray fluoroscopy device, an MRI device, an ultrasound device, a cone-beam CT device, a CT fluoroscopy device, an optical imaging device and electromagnetic imaging device.

According to some embodiments, the at least one processor of the system is configured to obtain one or more images from the imaging device.

According to some embodiments, the system may further include one or more of: a user interface, a display, a control unit, a computer, or any combination thereof.

According to some embodiments, there is provided a method for determining the actual position of a tip of a medical instrument within a body of a subject, the method includes:
obtaining one or more images of the medical instrument within the body of the subject;
detecting the medical instrument in the one or more images;
defining an end of the detected medical instrument in the one or more images;
determining a compensation value for the end of the medical instrument; and
determining the actual position of the tip of the medical instrument in the body of the subject, based on the determined compensation value.

According to some embodiments, the compensation value is one of a positive compensation value, a negative compensation value and no (zero) compensation.

According to some embodiments, the one or more images are obtained using an imaging system. According to some embodiments, the imaging system is selected from: a CT system, an X-ray fluoroscopy system, an MRI system, an ultrasound system, a cone-beam CT system, a CT fluoroscopy system, an optical imaging system and electromagnetic imaging system.

According to some embodiments, the method for determining the actual position of the tip of a medical instrument within the body of a subject further includes determining the position and/or orientation of the medical instrument relative to a coordinate system of the imaging system.

According to some embodiments, the method may further include displaying the one or more images to a user. In some embodiments, the one or more images include CT scans. According to some embodiments, the compensation value is determined based on an angle of the medical instrument about the right-left axis of the CT scans. According to some embodiments, the compensation value may be determined based on a look-up table.

According to some embodiments, the compensation value may be determined based on one of more of: the imaging system, the operating parameters of the imaging system, the type of medical instrument, the dimensions of the medical instrument, the angle of the medical instrument, the tissue in which the medical instrument resides or any combination thereof.

According to some embodiments, the actual position of the tip of the medical instrument is the actual 3D position of the tip of the medical instrument.

According to some embodiments, the method may be performed in real-time. According to some embodiments, the method may be performed continuously and/or in time lapses.

According to some embodiments, there is provided a method for planning a 3D trajectory for a medical instrument insertable into a body of a subject, the method includes:
calculating a first planar trajectory for the medical instrument from an entry point to a target in the body of the subject, based on a first image or a first set of image frames of a region of interest, the first image frame or first set of image frames pertaining to a first plane;
calculating a second planar trajectory for the medical instrument from the entry point to the target, based on a second image frame or a second set of image frames of a region of interest, the second image frame or second set of image frames pertaining to a second plane; and
superpositioning the first and second planar trajectories to determine the 3D trajectory for the medical instrument from the entry point to the target.

According to some embodiments of the method for calculating a 3D trajectory for a medical instrument insertable into a body of a subject, the first and second planes are perpendicular.

According to some embodiments of the method, the target and the entry point are manually defined by a user.

According to some embodiments, the method may further include defining at least one of the target and the entry point on the first or second image frames or sets of image frames, using image processing and/or machine learning algorithms.

According to some embodiments, there is provided a system for planning a 3D trajectory for a medical instrument insertable into a body of a subject, the system includes:
a processor configured to execute the method for calculating a 3D trajectory for a medical instrument insertable into a body of a subject as disclosed herein;
a monitor configured to display at least the first image frame or first set of image frames, the second image frame or set of image frames, the target, the entry point and the calculated first and second planar trajectories; and
a user interface configured to receive user input.

According to some embodiments, there is provided a method for updating in real- time a 3D trajectory of a medical instrument, the 3D trajectory extending from an insertion point to a target in the body of a subject, the method includes:
defining a real-time position of the target;
determining if the real-time position of the target deviates from a previous target position;
if it is determined that the real-time position of the target deviates from the previous target position:
   calculating a first 2D trajectory correction on a first plane;
   calculating a second 2D trajectory correction on a second plane; and
   determining the 3D trajectory correction for the tip by superpositioning the first and second 2D trajectory corrections.

According to some embodiments of the method for updating in real-time a 3D trajectory of a medical instrument, the first and second planes are perpendicular to each other.

According to some embodiments of the method, calculating the first and second 2D trajectory corrections utilizes an inverse kinematics algorithm.

According to some embodiments of the method, defining the real-time position of the target includes receiving user input thereof.

According to some embodiments of the method, defining the real-time position of the target includes automatically identifying the real-time position of the target, using image processing and/or machine learning algorithms.

According to some embodiments of the method, defining the real-time position includes real-time tracking the position of the target within the body.

According to some embodiments, there is provided a method for updating in real- time a 3D trajectory of a medical instrument, the 3D trajectory extending from an insertion point to a target in the body of a subject, the method includes:
defining a real-time position of the target;
defining a real-time position of the medical instrument;
determining if the real-time position of the target deviates from a previous target position and/or if the medical instrument deviates from a planned 3D trajectory based on the defined real-time position of the medical instrument;
if it is determined that the real-time position of the target deviates from the previous target position and/or that the medical instrument deviates from the planned 3D trajectory:
   calculating a first planar trajectory correction on a first plane;
   calculating a second planar trajectory correction on a second plane; and
   determining the 3D trajectory correction for the tip by superpositioning the first and second planar trajectory corrections.

According to some embodiments, there is provided a system for updating in real-time a 3D trajectory of a medical instrument, the 3D trajectory extending from an insertion point to a target in the body of a subject, the system includes:
a processor configured to execute a method for updating in real-time a 3D trajectory of a medical instrument;
a monitor configured to display the target, the insertion point and the calculated first and second planar trajectories on one or more image frames; and
a user interface configured to receive input from a user.

According to some embodiments, there is provided a method of steering a medical instrument toward a target within a body of a subject, the method includes:
calculating a planned 3D trajectory for the medical instrument from an entry point to a target in the body of the subject;
steering the medical instrument toward the target according to the planned 3D trajectory;
determining if at least one of: (i) a real-time position of the target deviates from a previous target position, (ii) a real-time position of the medical instrument deviates from the planned 3D trajectory, and (iii) one or more obstacles are identified along the planned 3D trajectory;
if it is determined that the real-time position of the target deviates from the previous target position, that the real-time position of the medical instrument deviates from the planned 3D trajectory, and/or that one or more obstacles are identified along the planned trajectory, updating the 3D trajectory of the medical instrument to facilitate the medical instrument reaching the target, and
   steering the medical instrument toward the target according to the updated 3D trajectory.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

### BRIEF DESCRIPTION OF THE DRAWINGS

Some exemplary implementations of the methods and systems of the present disclosure are described with reference to the accompanying drawings. In the drawings, like reference numbers indicate identical or substantially similar elements.

FIG. 1A shows a schematic perspective view of a device for inserting and steering a medical instrument into the subject body according to a planned and real-time updated 3D trajectory, according to some embodiments;

FIG. 1B shows a perspective view of an exemplary control unit of a system for inserting and steering a medical instrument into the body of a subject according to a planned and real-time updated 3D trajectory, according to some embodiments;

FIG. 2 shows an exemplary planned trajectory for a medical instrument to reach an internal target within the body of the subject, according to some embodiments;

FIG. 3A shows CT images of a subject (left-hand panel: axial plane; right-hand panel: sagittal plane), further showing the internal target, the insertion and steering device and potential obstacles;

FIG. 3B shows CT images of a subject (left-hand panel: axial plane; right-hand panel: sagittal plane), further showing the internal target, the insertion point, a linear trajectory for the medical instrument from the insertion point to the target, potential obstacles, the medical instrument and the insertion and steering device;

FIG. 3C shows CT images of a subject (left-hand panel: axial plane; right-hand panel: sagittal plane), further showing the internal target, the insertion point, a linear trajectory for the medical instrument from the insertion point to the target, a marked obstacle along the linear trajectory, the medical instrument and the insertion and steering device;

FIG. 3D shows CT images of a subject (left-hand panel: axial plane; right-hand panel: sagittal plane), further showing the internal target, the insertion point, a non-linear trajectory for the medical instrument from the insertion point to the target, a marked obstacle along the planned trajectory, the medical instrument and the insertion and steering device;

FIG. 4 shows a CT image of a subject showing a medical instrument inserted and steered within the body, the tip of which reaching an internal target, according to an updated trajectory of the medical instrument, wherein the updated trajectory is based on the real-time location of the target. Also shown is the original location of the target;

FIG. 5 shows a flow chart of steps in a method for planning and real-time updating a 3D trajectory of a medical instrument, according to some embodiments;

FIG. 6 shows a flow chart of steps in a method for determining the actual location of a tip of a medical instrument in images of a subject, according to some embodiments;

FIG. 7A shows CT images (left and right-hand panels) of lungs of a porcine, having a medical instrument (needle) steered thereto based on a planned and updated 3D trajectory, to reach a target (lung bifurcation);

FIG. 7B shows CT images (left and right-hand panels) of kidney tissue of a porcine, having a medical instrument (needle) inserted and steered to a target therewithin, based on a planned and updated 3D trajectory;

FIGS. 8A-8C show close-up views of a tip of a medical instrument in CT scans and the indicated actual locations thereof.

### DETAILED DESCRIPTION

The principles, uses and implementations of the teachings herein may be better understood with reference to the accompanying description and figures. Upon perusal of the description and figures present herein, one skilled in the art will be able to implement the teachings herein without undue effort or experimentation. In the figures, same reference numerals refer to same parts throughout.

In the following description, various aspects of the invention will be described. For the purpose of explanation, specific details are set forth in order to provide a thorough understanding of the invention. However, it will also be apparent to one skilled in the art that the invention may be practiced without specific details being presented herein. Furthermore, well-known features may be omitted or simplified in order not to obscure the invention.

According to some embodiments, there are provided systems, devices and methods for insertion and steering of a medical instrument in a subject's body wherein the steering of the medical instrument within the body of a subject is based on planning and real-time updating the 3D trajectory of the medical instrument (in particular, the end or tip thereof), within the body of the subject, to facilitate the safe and accurate reaching of the tip to an internal target region within the subject's body, by the most efficient and safe route. In further embodiments, there are provided systems, devices and methods allowing the precise determination of the actual location of the tip of the medical instrument within the body, to increase effectiveness, safety and accuracy of various related medical procedures.

In some embodiments, a medical device for inserting and steering a medical instrument into (and within) a body of a subject may include any suitable automated device. The automated steering device may include any type of suitable steering mechanism allowing or controlling the movement of an end effector (control head) at any one of desired movement angles or axis. In some embodiments, the automated inserting and steering device may have at least 3 degrees of freedom, at least 4 degrees of freedom, or at least five degrees of freedom (DOF).

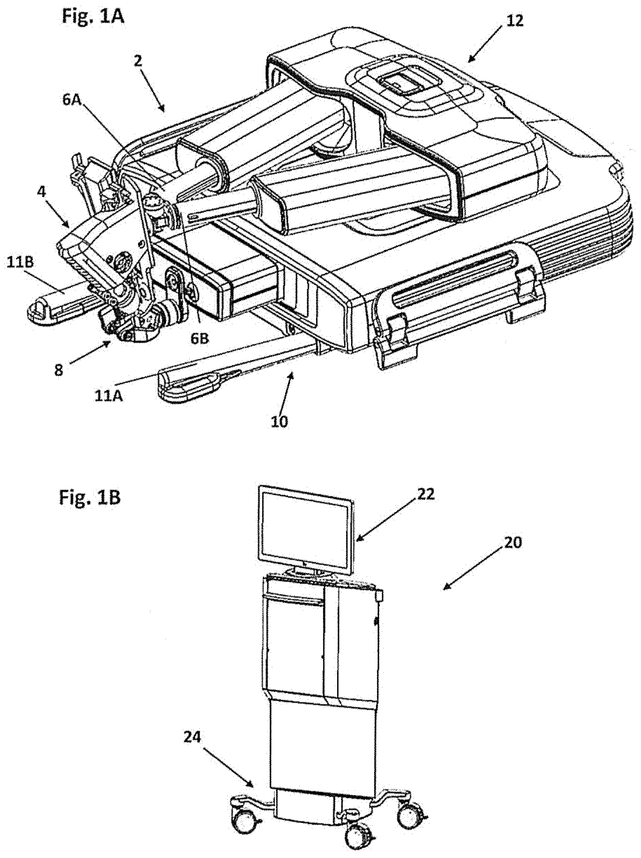

Reference is now made to FIG. 1A, which shows a schematic illustration of an exemplary device for inserting and steering a medical instrument in a body of a subject, based on a planned 3D trajectory, which may be updated in real-time, according to some embodiments. As shown in FIG. 1A, the insertion and steering device 2 may include a housing (also referred to as “cover”) 12 accommodating therein at least a portion of the steering mechanism. The steering mechanism may include at least one moveable platform (not shown) and at least two moveable arms 6A and 6B, configured to allow or control movement of an end effector (also referred to as “control head”) 4, at any one of desired movement angles or axis, as disclosed, for example, in co-owned U.S. Patent Application Publication No. 2019/290,372, to Arnold et al, which is incorporated herein by reference in its entirety. The moveable arms 6A and 6B may be configured as piston mechanisms. To the end 8 of control head 4, a suitable medical instrument (not shown) may be connected, either directly or by means of a suitable insertion module, such as the insertion module disclosed in co-owned U.S. Patent Application Publication No. 2017/258,489, to Galili et al, which is incorporated herein by reference in its entirety. The medical instrument may be any suitable instrument capable of being inserted and steered within the body of the subject, to reach a designated target, wherein the control of the operation and movement of the medical instrument is effected by the control head 4. The control head 4 may be controlled by a suitable control system, as detailed herein.

According to some embodiments, the medical instrument may be selected from, but not limited to: a needle, probe (e.g., an ablation probe), port, introducer, catheter (such as a drainage needle catheter), cannula, surgical tool, fluid delivery tool, or any other suitable insertable tool configured to be inserted into a subject's body for diagnostic and/or therapeutic purposes. In some embodiments, the medical tool includes a tip at the distal end thereof (i.e., the end which is inserted into the subject's body).

In some embodiments, the device 2 may have a plurality of degrees of freedom (DOF) in operating and controlling the movement the of the medical instrument along one or more axis and angles. For example, the device may have up to six degrees of freedom. For example, the device may have at least five degrees of freedom. For example, the device may have five degrees of freedom, including: forward-backward and left-right linear translations, front-back and left-right rotations, and longitudinal translation toward the subject's body. For example, the device may have six degrees of freedom, including the five degrees of freedom described above and, in addition, rotation of the medical instrument about its longitudinal axis.

In some embodiments, the device may further include a base 10, which allows positioning of the device on or in close proximity to the subject's body. In some embodiments, the device may be attached to the subject's body directly, or via a suitable mounting surface. In some embodiments, the device may be attached to the subject's body by being coupled to a mounting apparatus, such as the mounting base disclosed in co-owned U.S. Patent Application Publication No. 2019/125,397, to Arnold et al, or the attachment frame disclosed in co-owned International Patent Application Publication No. WO 2019/234748, to Galili et al, both of which are incorporated herein by reference in their entireties. In some embodiments, the device may be coupled/attached to a dedicated arm (stationary, robotic or semi-robotic) or base which is secured to the patient's bed, to a cart positioned adjacent the patient's bed or to an imaging device (if such is used), and held on the subject's body or in close proximity thereto, as described, for example, in U.S. Pat. Nos. 10,507,067 and 10,639,107, both to Glozman et al, and both incorporated herein by reference in their entireties.

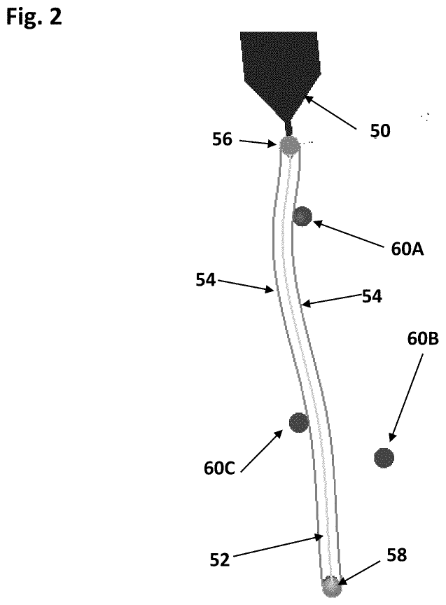

In some embodiments, the device further includes electronic components and motors (not shown) allowing the controlled operation of the device 2 in inserting and steering the medical instrument. In some exemplary embodiments, the device may include one or more Printed Circuit Board (PCB) (not shown) and electrical cables/wires (not shown) to provide electrical connection between the controller (described in connection with FIG. 2 hereinbelow) and the motors of the device and other electronic components thereof. In some embodiments, the housing 12 covers and protects, at least partially, the mechanical and electronic components of the device 2 from being damaged or otherwise compromised.

In some exemplary embodiments, the device may further include fiducial markers (or “registration elements”) disposed at specific locations on the device 2, such as registration elements 11A and 11B, for registration of the device to the image space, in image-guided procedures.

In some embodiments, the device is automated (i.e., a robot). In some embodiments, the medical instrument is configured to be removably coupleable to the device 2, such that the device can be used repeatedly with new medical instruments. In some embodiments, the automated device is a disposable device, i.e., a device which is intended to be disposed of after a single use. In some embodiments, the medical instruments are disposable. In some embodiments, the medical instruments are reusable.

According to some exemplary embodiments, there is provided, an automated device for inserting and steering a medical instrument into an internal target in a body of a subject, based on a planned and/or real-time updated 3D trajectory, to facilitate the reaching of the tip of the medical instrument to a desired internal target, the device includes a steering mechanism, which may include, for example, (i) at least one moveable platform; (ii) one or more piston mechanisms, each piston mechanism including: a cylinder, a piston, at least a portion of which is being positioned within the cylinder, and a driving mechanism configured to controllably propel the piston in and out of the cylinder, and (iii) an insertion mechanism configured to impart longitudinal movement to the medical instrument. In some embodiments, the distal ends of the pistons may be coupled to a common joint. In some embodiments, the cylinders, pistons and the common joint may all be located substantially in a single plane, allowing larger angular movement and thus a larger workspace for the device's control head and medical instrument, as disclosed in abovementioned U.S. Patent Application Publication No. 2019/290,372.

According to some embodiments, the device 2 may further include one or more sensors (not shown). In some embodiments, the sensor may be a force sensor. In some embodiments, the device does not include a force sensor. According to some embodiments, the device may include a virtual Remote Center of Motion located, for example, at a selected entry point on the body of the subject.

In some embodiments, the device 2 is operable in conjunction with a system for inserting and steering a medical instrument in a subject's body based on a planned and updated 3D trajectory of the medical instrument. In some embodiments, the system includes the steering and insertion device 2 as disclosed herein and a control unit configured to allow control of the operating parameters of the device.

In some embodiments, the system may include one or more suitable processors used for various calculations and manipulations, including, for example, but not limited to: determination/planning of a 3D trajectory of the medical instrument, updating in real-time the 3D trajectory, image processing, and the like. In some embodiments, the system may further include a display (monitor) which allows presenting of the determined and updated 3D trajectory, one or more obtained images or sets of images or image-views created from sets of images (between which the user may be able to scroll), operating parameters, and the like. The one or more processors may be implemented in the form of a computer (such as a PC, a laptop, a tablet, a smartphone, or any other processor-based device). In some embodiments, the system may further include a user interface (such as in the form of buttons, switches, keys, keyboard, computer mouse, joystick, touch-sensitive screen, extended reality (virtual reality, augmented reality and/or mixed reality) glasses, headset or goggles, and the like). The display and user interface 132 may be two separate components, or they may form together a single component. In some exemplary embodiments, the processor (for example, as part of a computer) may be configured to perform one or more of: determine (plan) the 3D trajectory (pathway) for the medical instrument to reach the target; update in real-time the 3D trajectory; present the planned and/or updated trajectory; control the movement (steering and insertion) of the medical instrument based on the pre-planned and/or updated 3D trajectory, by providing executable instructions (directly or via one or more controllers) to the device; determine the actual location of the medical instrument by performing required compensation calculations; receive, process and visualize on the display images obtained from the imaging system or image-views created from a set of images; and the like, or any combination thereof.

In some embodiments, the system may be configured to operate in conjunction with an imaging system, including, but not limited to: X-ray fluoroscopy, CT, cone beam CT, CT fluoroscopy, MRI, ultrasound, or any other suitable imaging modality. In some embodiments, the insertion and steering of the medical instrument based on a planned and real-time updated 3D trajectory of the medical instrument is image-guided.

According to some embodiments, the planned 3D trajectory of the medical instrument (in particular, the tip thereof) may be calculated, inter alia, based on input from the user, such as the entry point, target and, optionally, areas to avoid en route (obstacles), which the user marks on at least one of the obtained images. In some embodiments, the processor may be further configured to identify and mark the target, the obstacles and/or the insertion/entry point.

According to some embodiments, the system may further include a controller (for example, a robot controller), which controls the movement of the insertion and steering device and the steering of the medical instrument towards the target within the subject's body. In some embodiments, at least a portion of the controller may be embedded within the device, and/or within the computer. In some embodiments, the controller may be a separate component.

Reference is now made to FIG. 1B, which schematically illustrates a control unit (workstation) 20 of a system for insertion and steering of a medical instrument based on the planned and real-time updated trajectory of the tip of the medical instrument, according to some embodiments. The control unit 20 may include a display/monitor 22 and a user interface (not shown). The control unit may further include a processor (for example, in the form of a PC). According to some embodiments, the control unit 20 may further include a controller (for example, a robot controller), which controls the movement of the insertion and steering device and the steering of the medical instrument towards the target within the subject's body. The control unit/workstation may be portable (for example, having or being placed on a movable platform 24). As detailed above, the control unit is configured to physically and/or functionally interact with the insertion and steering device, to determine and control the operation thereof.

Reference is now made to FIG. 2, which schematically illustrates trajectory planning, according to some embodiments. As shown in FIG. 2, a trajectory 52 is planned between an entry point 56 and an internal target 58. The planned trajectory 52 takes into account various variables, including, but not limited to: the type of the medical instrument to be inserted, the dimensions of the medical instrument (e.g., length, gauge), the tissues through which the medical instrument is inserted, the position of the target, the size of the target, the insertion point, the angle of insertion, and the like, or any combination thereof. In some embodiments, further taken into account in determining the trajectory are various obstacles (shown as obstacles 60A-60C), which may be identified along the path and should be avoided, to prevent damage to the neighboring tissues and/or to the medical instrument. According to some embodiments, safety margins 54 are marked along the planned trajectory 52, to ensure a minimal distance between the trajectory 52 and potential obstacles en route. The width of the safety margins may be symmetrical in relation to the trajectory 52. The width of the safety margins may be asymmetrical in relation to the trajectory 52. According to some embodiments, the width of the safety margins 54 is preprogrammed. According to some embodiments, the width of the safety margins may be recommended by the processor based on data obtained from previous procedures, using machine learning capabilities. According to other embodiments, the width of the safety margins 54 may be determined and/or adjusted by the user. Further shown in FIG. 2 is the end (e.g., control head) 50 of the insertion and steering device, to which the medical instrument (not shown in FIG. 2) is coupled, as virtually displayed on the monitor, to indicate its position and orientation. The presented trajectory shown in FIG. 2, is in a planar trajectory (i.e., two dimensional) and it may be used in determining the 3D trajectory by being superpositioned with a second planar trajectory, which may be planned on a plane perpendicular to the plane of the trajectory shown in FIG. 2.

According to some embodiments, as detailed herein, the planned 3D trajectory and/or the updated 3D trajectory may be calculated by determining a pathway on each of two planes, which are superpositioned to form a three-dimensional trajectory. In some embodiments, the two planes may be perpendicular to one another. According to some embodiments, the steering of the medical instrument is carried out in a 3D space, wherein the steering instructions are determined on each of two planes, which are superpositioned to form the steering in the three-dimensional space. In some embodiments, the planned 3D trajectory and/or the updated 3D trajectory may be calculated by calculating a pathway on each of two planes, and then superpositioning the two planar trajectories to form a three-dimensional trajectory. In some embodiments, the planned 3D trajectory and/or an updated 3D trajectory may be calculated on two planes, which may be at least partially superpositioned to form a 3D trajectory. In some embodiments, a planned 3D trajectory and/or an updated 3D trajectory may be calculated based on a combination or superpositioning of 2D trajectories calculated on several intersecting planes.

According to some embodiments, the 3D trajectory may include any type of trajectory, including a linear trajectory or a non-linear trajectory having any suitable degree of curvature.

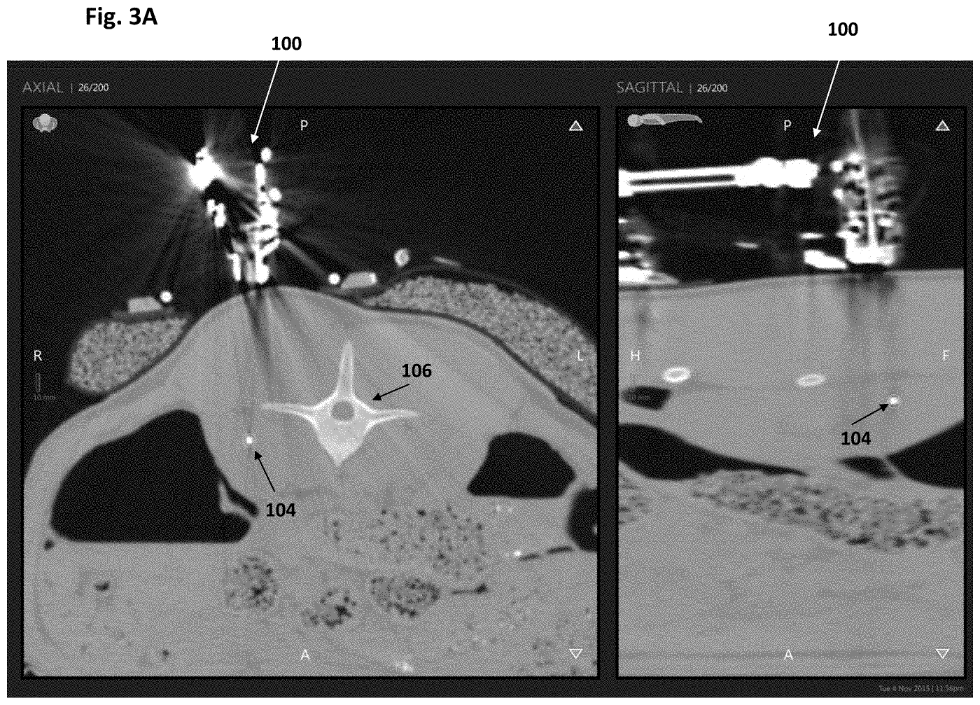

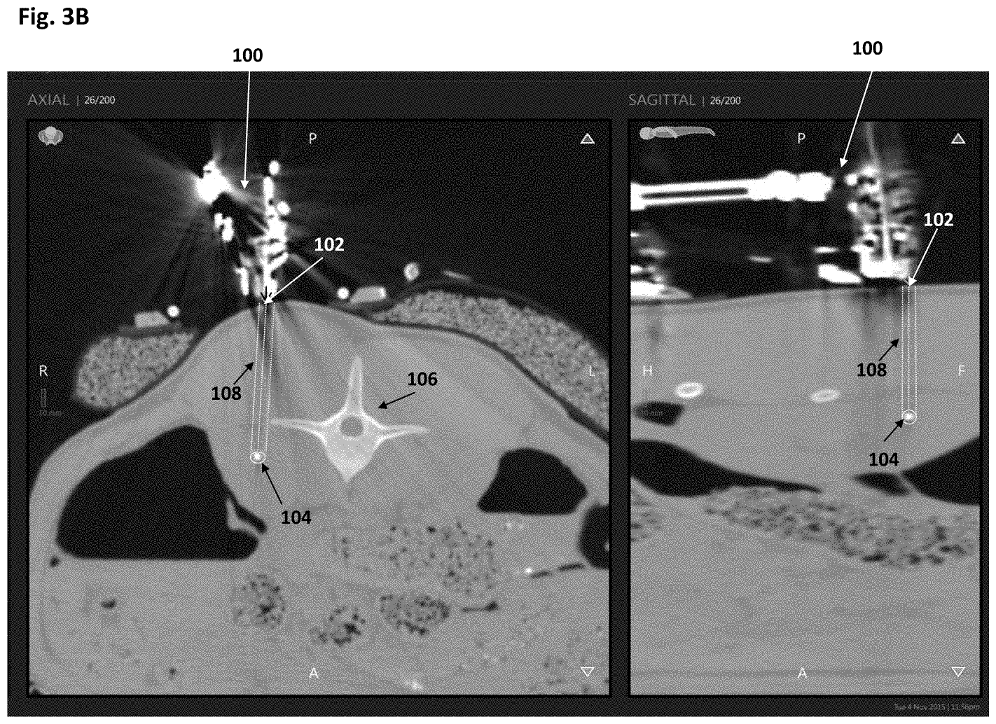

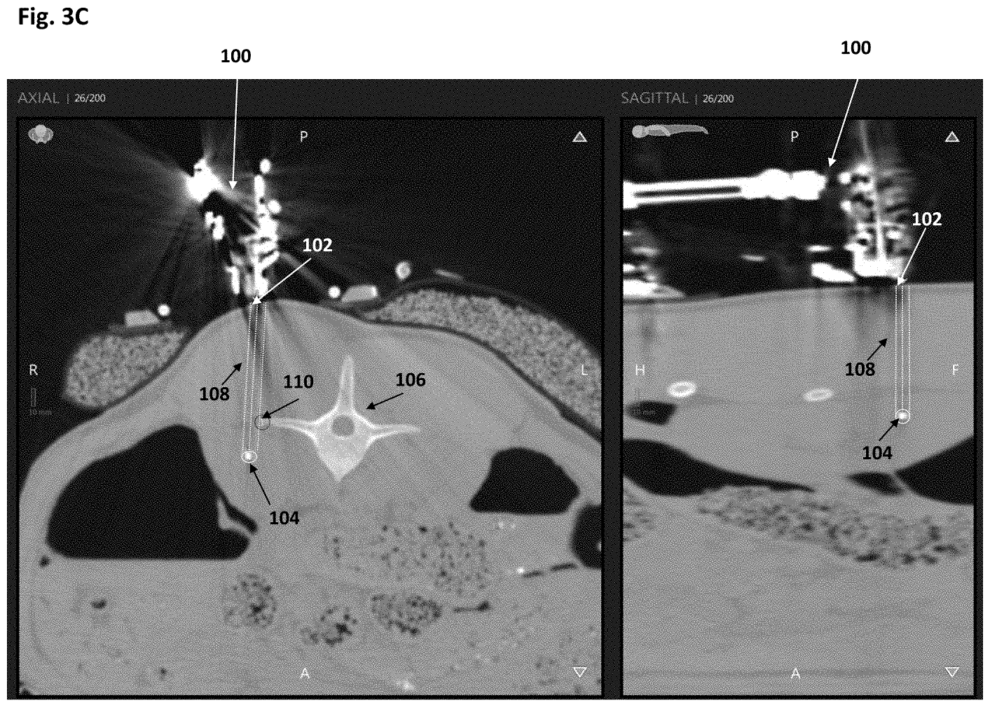

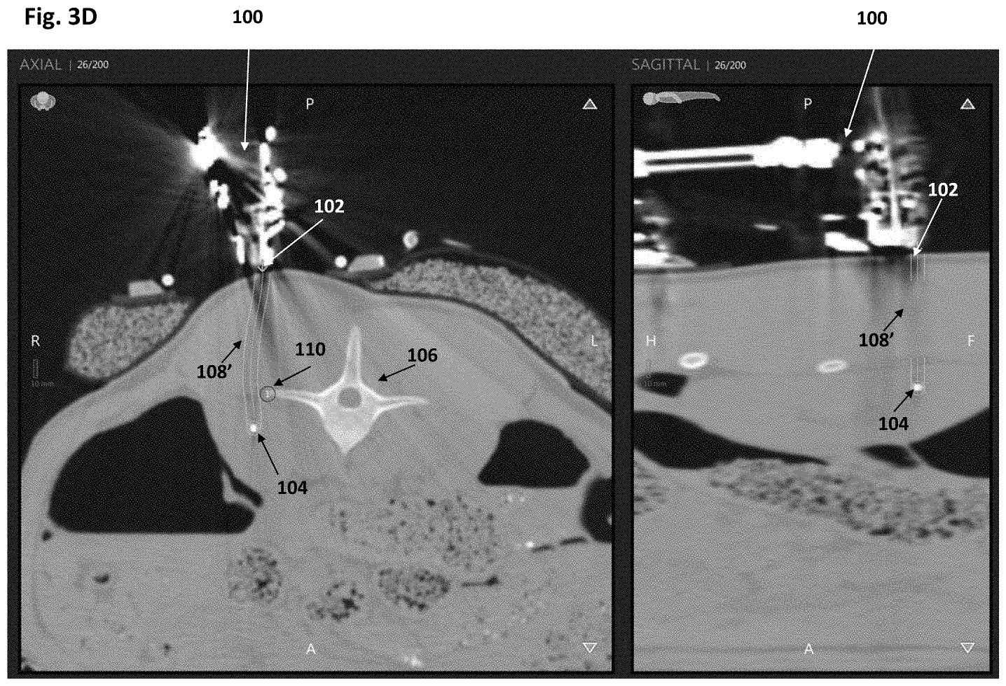

Reference is now made to FIGS. 3A-3D, which show exemplary 3D trajectory planning for insertion and steering of a medical instrument towards a target, on CT image-views, according to some embodiments. Shown in FIG. 3A are CT image-views of a subject, depicting at the left-hand panel an axial plane view and on the right-hand panel a sagittal plane view. Also indicated in the figure is an internal target 104 and the insertion and steering device 100. Also indicated is a vertebra 106, which may be identified as an obstacle which the medical instrument should avoid. In FIG. 3B, which shows the CT image-views of FIG. 3A, the insertion point 102 is indicated. Consequently, according to some embodiments, a linear trajectory 108 between the entry point 102 and the internal target 104 is then calculated and displayed on each of the two views (for example, axial plane view and sagittal plane view). Typically, a linear trajectory is preferred, thus, if the displayed linear trajectory does not pass in close proximity to any potential obstacles, then the linear trajectory is determined as the planned trajectory for the insertion procedure. In FIG. 3C, a transverse process 110 of vertebra 106 is detected in close proximity to the calculated linear trajectory, and is identified and marked, in this example on the axial plane view, to allow considering the obstacle when planning the trajectory for the procedure. In FIG. 3D, the trajectory is re-calculated to result in a non-linear trajectory 108′, which allows avoiding contacting the obstacle 110. According to some embodiments, the planned trajectory is not calculated until potential obstacles are marked on the image-view/s, either manually or automatically, or until the user confirms that there are no potential obstacles, and/or until the user manually initiates trajectory calculation. In such embodiments, if there are obstacles which necessitate a non-linear trajectory, an interim linear trajectory, similar to linear trajectory 108 of FIG. 3B, is not calculated and/or displayed. According to some embodiments, a maximal allowable curvature level may be pre-set for the calculation of the non-linear trajectory. The maximal curvature threshold may depend, for example, on the trajectory parameters (e.g., distance between the entry point and the target) and on the type of instrument intended to be used in the procedure and its characteristics (for example, type, diameter (gauge), and the like). The two calculated non-linear 2D trajectories may then be superpositioned to form the non-linear 3D trajectory which is used for steering the medical instrument from the entry point 102 to the target 104. As further detailed below, the planned 3D trajectory can be updated in real-time based on the real-time position of the medical instrument (for example, the tip thereof) and/or the real-time position of the target and/or the obstacle/s.

According to some embodiments, the target 104, insertion point 102 and, optionally, obstacle/s 110 are marked manually by the user. According to other embodiments, the processor may be configured to identify and mark at least one of the target, the insertion point and the obstacle/s, and the user may, optionally, be prompted to confirm or adjust the processor's proposed markings. In such embodiments, the target and/or obstacle/s may be identified using known image processing techniques and/or machine learning tools (algorithms) based on data obtained from previous procedures, and the entry point may be suggested based solely on the obtained images, or, alternatively or additionally, also on data obtained from previous procedures using machine learning capabilities.

According to some embodiments, the trajectory may be calculated based solely on the obtained images and the marked locations of the entry point, target and, optionally, obstacle/s. According to other embodiments, the trajectory may be calculated based also on data obtained from previous procedures, using machine learning capabilities. According to some embodiments, once the planned trajectory has been determined, checkpoints along the trajectory may be set. The checkpoints may be manually set by the user, or they may be automatically set by the processor, as described in further detail hereinbelow.

It can be appreciated that although axial and sagittal views are shown in FIGS. 3A-3D, views pertaining to different planes or orientations (e.g., coronal, pseudo axial, pseudo sagittal, pseudo coronal, etc.) or additionally generated views (e.g., trajectory view, tool view, 3D view, etc.), may be used in order to perform and/or display the trajectory planning and/or updating.

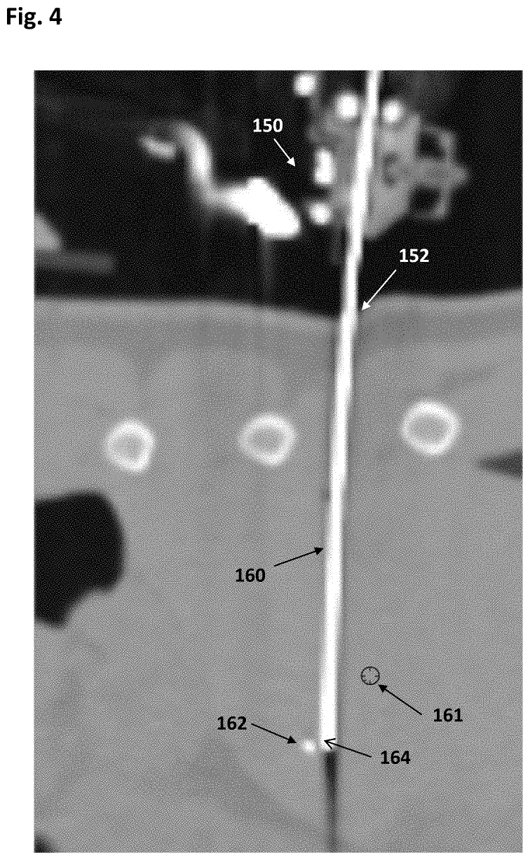

Reference is now made to FIG. 4, which shows a CT image of a subject, showing the medical instrument inserted and steered within the body, having the tip thereof reaching an internal target, according to an updated trajectory of the medical instrument, wherein the updated trajectory is based on the real-time location of the target. As shown in FIG. 4, the medical instrument 160 is inserted and steered by the insertion and steering device 150. The medical instrument 160 (e.g., an introducer or a needle) is inserted from entry point 152 and is steered along a 3D trajectory towards the target. The planned trajectory was calculated to allow the medical instrument to reach the target at its initial location 161. However, the 3D trajectory was updated in real-time, to reflect changes in the real-time position 162 of the target, to allow the accurate steering of the tip 164 of the medical instrument to the actual, real-time location 162 of the target.

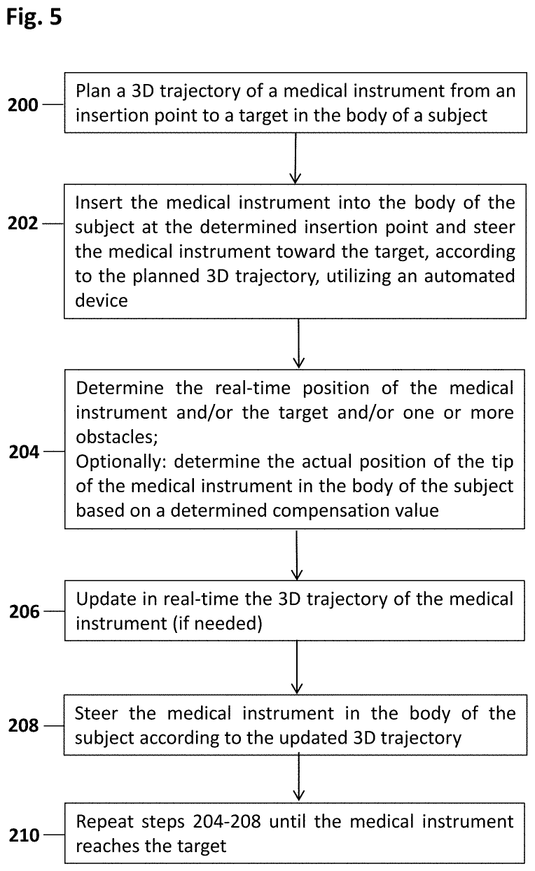

Reference is now made to FIG. 5, which illustrates steps in a method for planning and updating a 3D trajectory of a medical instrument to an internal target in a body of a subject, according to some embodiments. At step 200, the 3D trajectory of the medical instrument is planned from an insertion point on the body of the subject to an internal target. In some embodiments, the planned 3D trajectory may be obtained by planning a route on each of two planes and superpositioning the two 2D routes on said planes, at their intersection line, to form the planned 3D trajectory. In some exemplary embodiments, the two planes are perpendicular. The planned route may take into account various parameters, including but not limited to: type of medical instrument, type of imaging modality (such as, CT, CBCT, MRI, X-RAY, CT Fluoroscopy, Ultrasound and the like), insertion point, insertion angle, type of tissue(s), location of the internal target, size of the target, obstacles along the route, milestone points (“secondary targets” through which the medical instrument should pass) and the like, or any combination thereof. In some embodiments, at least one of the milestone points may be a pivot point, i.e., a predefined point along the trajectory in which the deflection of the medical instrument is prevented or minimized, to maintain minimal pressure on the tissue (even if this results in a larger deflection of the instrument in other parts of the trajectory). In some embodiments, the planned trajectory is an optimal trajectory based on one or more of these parameters.

Next, at step 202, the medical instrument is inserted into the body of the subject at the designated (selected) entry point and steered (in a 3D space) towards the predetermined target, according to the planned 3D trajectory. As detailed herein, the insertion and steering of the medical instrument is facilitated by an automated device for inserting and steering, such as, for example, device 2 of FIG. 1A.

At step 204, the real-time location/position (and optionally the orientation) of the medical instrument (e.g., the tip thereof) and/or the real-time 3D actual trajectory (i.e.

movement or steering) of the medical instrument and/or the real-time location of one or more obstacles and/or the location of newly identified one or more obstacles along the trajectory and/or the real-time location of one or more of the milestone points (“secondary targets”) and/or the real-time location of the target are determined. Each possibility is a separate embodiment. In some embodiments, the determination of any of the above may be performed manually by the user. In some embodiments, the determination of any of the above may be performed automatically by one or more processors. In the latter case, the determination may be performed by any suitable methods known in the art, including, for example, using suitable image processing techniques and/or machine learning (or deep learning) algorithms, using data collected in previous procedures (procedures previously performed). Step 204 may optionally further include correcting the determined location of the tip of the medical instrument, to compensate for deviations due to imaging artifacts, in order to determine the actual location of the tip. Determining the actual location of the tip prior to updating the 3D trajectory, can in some embodiments vastly increase the accuracy of the procedure. The determination of the actual location of the tip by calculating the required compensation may be performed as further detailed and exemplified herein below. In some embodiments, the determination may be performed at any spatial and/or temporal distribution/pattern and may be continuous or at any time (temporal) or space (spatial) intervals. In some embodiments, the procedure may halt at the spatio/temporal intervals to allow processing, determining, changing and/or approving continuation of the procedure. For example, the determination may be performed at one or more checkpoints. In some embodiments, the checkpoints may be predetermined and/or determined during the steering procedure. In some embodiments, the checkpoints may include spatial checkpoints (for example, regions or locations along the trajectory, including, for example, specific tissues, specific regions, length or location along the trajectory (for example, every 20-50 mm), and the like). In some embodiments, the checkpoints may be temporal checkpoints, i.e., a checkpoint performed at designated time points during the procedure (for example, every 2-5 seconds). In some embodiments, the checkpoints may include both spatial and temporal check points. In some embodiments, the checkpoints may be spaced apart, including the first checkpoint from the entry point and the last checkpoint from the target, at an essentially similar distance along the planned 3D trajectory. According to some embodiments, the checkpoints may be manually set by the user. According to some embodiments, the checkpoints may be automatically set by the processor, using image processing or computer vision algorithms, based on the obtained images and the planned trajectory and/or also on data obtained from previous procedures using machine learning capabilities. In such embodiments, the user may be required to confirm the checkpoints recommended by the processor or adjust their location/timing. Upper and/or lower interval thresholds between checkpoints may be predetermined. For example, the checkpoints may be automatically set by the processor at, for example, about 20 mm intervals, and the user may be permitted to adjust the distance between each two checkpoints (or between the entry point and the first checkpoint and/or between the last checkpoint and the target) such that the maximal distance between them is, for example, about 30 mm and/or the minimal distance between them is about 3 mm. Once the real-time location of any of the above parameters, or at least the real-time position of the target is determined, it is determined if there is a deviation in one or more of the abovementioned parameters from the initial/expected position and/or from the planned 3D trajectory, and if a deviation is determined, then, at step 206, the 3D trajectory is updated. The deviation may be determined compared to a previous time point or spatial point, as detailed above. In some embodiments, if a deviation in one or more of the abovementioned parameters is detected, the deviation is compared with a respective threshold, to determine if the deviation exceeds the threshold. The threshold may be, for example, a set value or a percentage reflecting a change in a value. The threshold may be determined by the user. The threshold may be determined by the processor, for example based on data collected in previous procedures and using machine learning algorithms. If deviation is detected, or if the detected deviation exceeds the set threshold, the 3D trajectory may be updated by updating the route, according to the required change, in each of two planes (for example, planes perpendicular thereto) and thereafter superpositioning the two updated 2D routes on the two (optionally perpendicular) planes to form the updated 3D trajectory. In some embodiments, the updated route on each of the two planes may be performed by any suitable method, including, for example, utilizing a kinematics model. In some embodiments, if the real-time location of the medical instrument indicates that the instrument has deviated from the planned 3D trajectory, the user may add and/or reposition one or more of the checkpoints along the planned trajectory, to direct the instrument back to the planned trajectory. In some embodiments, the processor may prompt the user to add and/or reposition checkpoint/s. In some embodiments, the processor may recommend to the user specific position/s for the new and/or repositioned checkpoints. Such a recommendation may be generated using image processing techniques and/or machine learning algorithms.

As detailed in step 208, the steering of the medical instrument is then continued, in a 3D space, according to the updated 3D trajectory, to facilitate the tip of the instrument reaching the internal target (and secondary targets along the trajectory, if such are required). It can be appreciated, that if no deviation in the abovementioned parameters was detected, the steering of the medical instrument can continue according to the planned 3D trajectory.

As indicated in step 210, steps 204-208 may be repeated for any number of times, until the tip of the medical instrument reaches the internal target, or until a user terminates the procedure. In some embodiments, the number of repetitions of steps 204-208 may be predetermined or determined in real-time, during the procedure. According to some embodiments, at least some of the steps (or sub-steps) are performed automatically. In some embodiments, at least some of the steps (or sub-steps) may be performed manually, by a user. According to some embodiments, one or more of the steps are performed automatically. According to some embodiments, one or more of the steps are performed manually. According to some embodiments, one or more of the steps are supervised manually and may proceed after being approved by user.

According to some embodiments, the 3D trajectory planning is a dynamic planning, allowing automatically predicting changes (for example, predicted target change), difficulties (for example, sensitive areas), obstacles (for example, undesired tissue), milestones, and the like, and adjusting the steering of the medical instrument accordingly, in fully automated or at least semi-automated manner. In some embodiments, the dynamic planning proposes a planned and/or updated 3D trajectory to a user for confirmation prior to proceeding with any of the steps. According to some embodiments, the 3D trajectory planning is a dynamic planning, taking into consideration expected cyclic changes in the position of the target, obstacles, etc., resulting from the body motion during the breathing cycle, as described, for example, in co-owned U.S. Pat. No. 10,245,110, to Shochat, which is incorporated herein by reference in its entirety. Such dynamic planning may be based on sets of images obtained during at least one breathing cycle of the subject (e.g., using a CT system), or based on a video generated during at least one breathing cycle of the subject (e.g., using a CT fluoroscopy system or any other imaging system capable of continuous imaging).

According to some embodiments, the steering of the medical instrument to the target is achieved by directing the medical instrument (for example, the tip of the medical instrument) in a 3D space, to follow, in real-time, the planned 3D trajectory, which may be updated in real-time, during the procedure, as needed.

According to some embodiments, the term “real-time 3D trajectory” relates to the actual movement/steering/advancement of the medical instrument in the body of the subject.

According to some exemplary embodiments, the 3D trajectory planning and updating using the systems disclosed herein is facilitated using any suitable imaging device. In some embodiments, the imaging device is a CT imaging device. In some embodiments, the planning and/or real-time updating of the 3D trajectory is performed based on CT images of the subject obtained before and/or during the procedure.

According to some embodiments, when utilizing various imaging modalities in the procedure, inherent difficulties may arise in identifying the actual location of the tip of the medical instrument. In some embodiments, the accurate orientation and position of the tool are important for high accuracy steering. Further, by determining the actual position of the tip, safety is increased, as the medical instrument is not inserted beyond the target or beyond what is defined by the user. Depending on the imaging modality, the tissue, and the type of medical instrument, artifacts which obscure the actual location of the tip can occur.

For example, when utilizing CT imaging, streaks and dark bands due to beam hardening can occur, which result in a “dark” margin at the end of the scanned instrument. The voxels at the end of the medical instrument may have very low intensity levels even if the actual medium or adjacent objects would normally have higher intensity levels. Additionally, point spread function (PSF) can occur in which the visible borders of the medical instrument are extended beyond their actual boundaries. Such artifacts can depend on the object's materials, size, and medical instrument angle relative to the CT, as well as on the scan parameters (FOV, beam power values) and reconstruction parameters (kernel and other filters).

Thus, depending on the type of the medical instrument, the imaging modality and/or the tissue, the tip position may not be easily visually detected, and in some cases, the determination may vastly deviate, for example by over 2-3 mm.

According to some embodiments, there is thus a need to compensate for such artifacts and inaccuracies to determine the actual location of the tip.

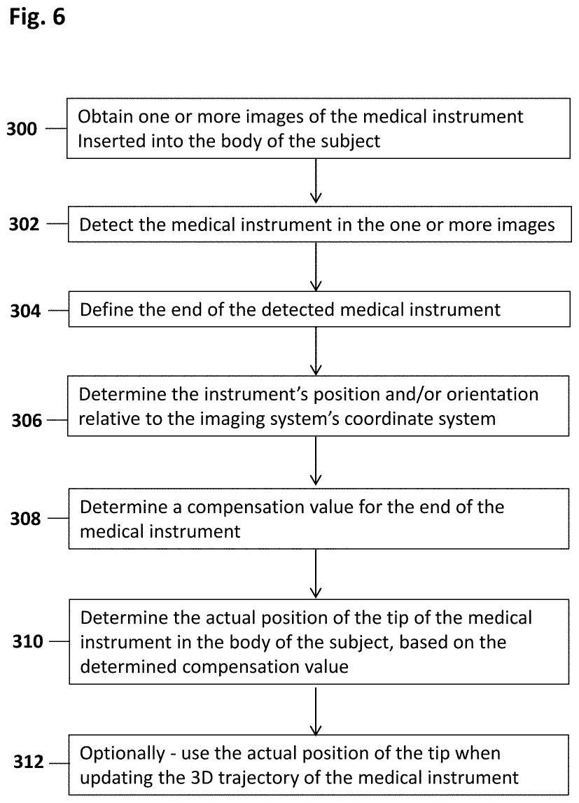

Reference is now made to FIG. 6, which details steps in a method for determining the actual position of a tip of a medical instrument, according to some embodiments. As shown in FIG. 6, at step 300, one or more images of the medical instrument within the subject's body are obtained. For example, the images may be CT images, or images obtained using any other suitable imaging modality, such as ultrasound, MRI, etc. At step 302, the medical instrument is detected in the one or more images, whereby the tip position is not accurately known. In step 304, the end of the detected medical instrument is defined. Defining the end of the medical instrument may take into account the max-gradient of the voxels' intensity between the medical instrument and its surroundings along the medical instrument object center line, in order to determine the relative point on or along the medical instrument from which the compensation is to be executed. This is then followed by step 306, in which the instrument's orientation and/or position relative to the imaging system's coordinate system is calculated. For example, in instances wherein the utilized imaging modality is a CT system, the instrument's angle about the CT's Right-Left axis may be calculated. Next, at step 308, a suitable compensation value for the correction of the actual position of tip of the medical instrument is determined. In some embodiments, the compensation value may be obtained based on any of the abovementioned imaging, tissue and/or medical tool parameters. In some exemplary embodiments, the compensation value may be obtained from a suitable look-up table. In some embodiments, the compensation value may be positive (if the actual tip position is past the visible end of the medical instrument) or negative (if the actual tip position is before the visible end of the instrument). Thus, in step 310, the actual position of the tip is determined accordingly, by said determined compensation/corrections.

According to some embodiments, the determination of the actual position of the tip is performed such as to result in determination of the actual 3D location of the tip, which may optionally be further presented to the user. In some embodiments, the determination of the actual location of the tip may be performed in 2D on two planes (that may, in some examples, be perpendicular), and the two determined locations are then superpositioned to provide the actual 3D position of the tip.

In optional step 312, the determined actual position of the tip can be used when updating the 3D trajectory of the medical instrument. For example, determining the actual position of the tip, as described above, may be an implementation, at least in part, of step 204 in the method described in FIG. 5 hereinabove.

According to some embodiments, the compensation value may depend on one or more parameters including, for example, instrument type, instrument dimensions (e.g., length), tissue, imaging modality, insertion angle, medical procedure, internal target, and the like. Each possibility is a separate embodiment.

In some embodiments, the methods provided herein allow determining the actual and relatively exact location of the tip, at below visualized pixel size level.

In some embodiments, the determination of the actual position of the tip may depend on the desired/required accuracy level, which may depend on several parameters, including, for example, but not limited to: the clinical indication (for example, biopsy vs. fluid drainage); the target size; the lesion size (for a biopsy procedure, for example); the anatomical location (for example, lungs/brain v. liver/kidneys); the 3D trajectory (for example, if it passes near delicate organs, blood vessels, etc.); and the like, or any combination thereof.

According to some exemplary embodiments, when CT imaging modality is used, the compensation value may depend, inter alia, on scanning parameters (helical vs axial), reconstruction parameters/kernel, Tube current (mA), Tube voltage (kV), insertion angle of the medical instrument relative to the CT right-left axis, CT manufacturers metal artifact's filtering, and the like. Each possibility is a separate embodiment.

According to some embodiments, the determination/correction of the actual location of the tip may be performed in real-time. According to some embodiments, the determination/correction of the actual location of the tip may be performed continuously and/or in time lapses on suitable images obtained from various imaging modalities.

Implementations of the systems and devices described above may further include any of the features described in the present disclosure, including any of the features described hereinabove in relation to other system and device implementations.

It is to be understood that the terms proximal and distal as used in this disclosure have their usual meaning in the clinical arts, namely that proximal refers to the end of a device or object closest to the person or machine inserting or using the device or object and remote from the patient, while distal refers to the end of a device or object closest to the patient and remote from the person or machine inserting or using the device or object.

It is to be understood that although some examples used throughout this disclosure relate to systems and methods for insertion of a needle into a subject's body, this is done for simplicity reasons alone, and the scope of this disclosure is not meant to be limited to insertion of a needle into the subject's body, but is understood to include insertion of any medical tool/instrument into the subject's body for diagnostic and/or therapeutic purposes, including a port, probe (e.g., an ablation probe), introducer, catheter (e.g., drainage needle catheter), cannula, surgical tool, fluid delivery tool, or any other such insertable tool.

In some embodiments, the terms “medical instrument” and “medical tool” may be used interchangeably.

In some embodiments, the terms “image”, “image frame”, “scan” and “slice” may be used interchangeably.

In some embodiments, the terms “user”, “doctor”, “physician”, “clinician”, “technician”, “medical personnel” and “medical staff” are used interchangeably throughout this disclosure and may refer to any person taking part in the performed medical procedure.

It can be appreciated that the terms “subject” and “patient” may refer either to a human subject or to an animal subject.

In the description and claims of the application, the words “include” and “have”, and forms thereof, are not limited to members in a list with which the words may be associated.

Unless otherwise defined, all technical and scientific terms used herein have the same meaning as commonly understood by one of ordinary skill in the art to which this disclosure pertains. In case of conflict, the patent specification, including definitions, governs. As used herein, the indefinite articles “a” and “an” mean “at least one” or “one or more” unless the context clearly dictates otherwise.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although steps of methods according to some embodiments may be described in a specific sequence, methods of the disclosure may include some or all of the described steps carried out in a different order. The methods of the disclosure may include a few of the steps described or all of the steps described. No particular step in a disclosed method is to be considered an essential step of that method, unless explicitly specified as such.

The phraseology and terminology employed herein are for descriptive purpose and should not be regarded as limiting. Citation or identification of any reference in this application shall not be construed as an admission that such reference is available as prior art to the disclosure. Section headings are used herein to ease understanding of the specification and should not be construed as necessarily limiting.

### EXAMPLES

### Example 1

### Insertion and Steering of a Medical Tool to an Internal Target, Based on Planned and Real-Time Updated 3D Trajectory

An insertion and steering system essentially as disclosed herein was used to automatically insert and steer a needle to an internal target through various tissues, based on a planned and then updated 3D trajectory of the tip of the needle.

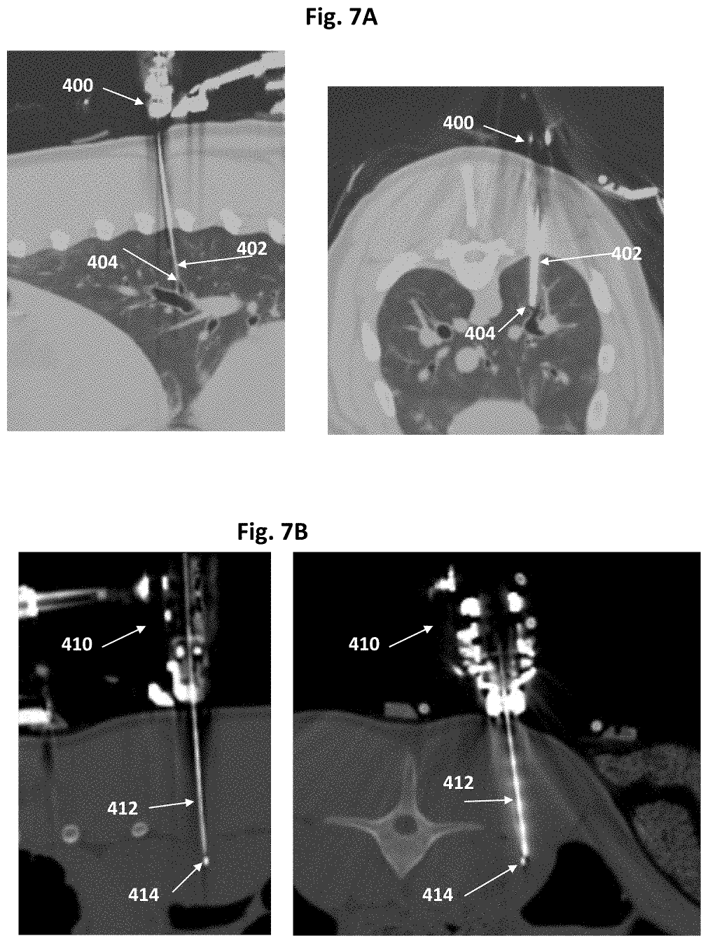

Shown in FIG. 7A (left and right-hand panels) are CT images of lungs of a porcine subject, wherein the medical instrument (needle 402) was inserted and steered based on a planned 3D trajectory, which was updated in real-time, to reach a target (lung bifurcation 404). Further shown is the insertion and steering device 400. The 3D trajectory from the insertion point to the target was at a length of about 103 mm.

Shown in FIG. 7B (left and right-hand panels) are CT images of kidney tissue of a porcine subject, wherein the medical instrument (needle 412) was inserted and steered based on a planned 3D trajectory, which was updated in real-time, to reach a target 414. Further shown is the insertion and steering device 410. The parameters of the 3D trajectory were a length of about 72 mm and the target size was 0.6 mm diameter×3 mm length.

The results presented in FIGS. 7A-7B demonstrate the advantageous accurate reaching of the medical instrument to a specific internal target in a safe and accurate manner, wherein the steering of the medical instrument (needle) in the body of the subject is performed automatically by a steering device, based on the real-time updating of the 3D trajectory of the tip of the needle to the target.

### Example 2

### Determining the Actual Location of a Tip of a Medical Instrument in CT Scans

In this example, the actual location of the tip of a medical instrument, such as needle, is determined, based on applying compensation to the detected location performed on CT images.

The medical tool (needle) insertion angle about the CT Right-Left axis can be between −80 to 80 degrees (0 degrees is when the entire needle is in one axial slice of the CT scan).

As detailed above herein, of the wide range of visual artifacts in CT scans, two types are of interest in relation to medical instruments, such as metal-needle like objects:

1. Streaks and dark bands due to beam hardening—‘dark’ margin at the end of the scanned medical instrument: The voxels at the end of the tool/needle may have very low intensity levels even if the actual medium or adjacent objects would normally have higher intensity levels.

2. PSF—point spread function—the medical instrument visible borders are extended beyond their actual boundaries.

These artifacts' effects can depend on the object's materials, size, and angle vis-a-vis the CT, as well as the scan parameters (FOV, beam power values) and the reconstruction parameters (kernel). In addition, different CT vendors may use different filters to compensate for such artifacts. These filters are also part of the artifacts' effect.

In order to compensate for these artifacts, the actual (real) location of the tip may be determined based on an instrument position compensation “look-up” table, which corresponds to the imaging method (CT in this example), and the medical instrument used. The compensation is relative to what is defined as the instrument's edge/end in an image. Thus, the defined instrument's edge/end, along with the compensation value from the “look-up” table, compose together the mechanism for determining the accurate tip position.

For example, the tip compensation may be determined based on the angle of the medical instrument about the CT Right-Left axis. The compensation may be positive compensation, no compensation and negative compensation—for the same tool depending on its angle about the CT Right-Left axis.

A “look-up” table may be obtained by testing various medical instrument types in a dedicated measuring device (jig) being CT scanned in a variety of angles (about the Right-Left axis). The measuring device provides the ground truth for the exact tip position. The measurements can be repeated for different scan parameters and reconstruction parameters.

An exemplary look up table 1 is presented below:

TABLE 1

| Angles | Corrections [mm] |
|---|---|
| 0 to +/−12 | 1.5 |
| 12 to 20 | 1.0 |
| −12 to −20 | 1.0 |
| 20 to 40 | 0.0 |
| −20 to −40 | 0.0 |
| 40 to 60 | −0.2 |
| −40 to −60 | −0.2 |
| 60 to 80 | 0.0 |
| −60 to −80 | 0.0 |

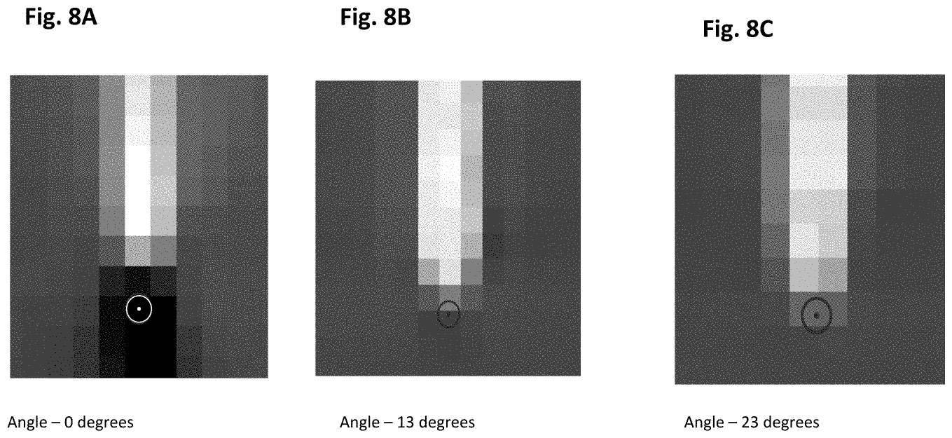

FIGS. 8A-C show close-up views of the tip of a needle as seen in CT scans during testing carried out in order to obtain a “look-up” table for that specific needle type. The encircled dots show the actual (physical) tip location (ground truth), based on physical registration between a needle tip position measuring device and the CT images. The millimetric distances mentioned below are the distances between the voxels with the lowest intensity (marking needle image edge) to the actual tip position.

FIG. 8A—positive compensation of about 1.27 (mm) (angle 0 degrees);

FIG. 8B—no compensation needed (angle 13 degrees);

FIG. 8C—negative compensation of about 0.29 mm (angle 23 degrees).

Thus, the results presented herein demonstrate the ability to accurately determine the actual location of the tip of the medical instrument, based on corresponding compensation values.

1-42. (canceled)

43. A method of steering a medical instrument toward a target within a body of a subject, the method comprising:
calculating a planned 3D trajectory for the medical instrument from an entry point to a target in the body of the subject;
steering the medical instrument toward the target according to the planned 3D trajectory;
determining if a real-time position of the target deviates from a previous target position;
if it is determined that the real-time position of the target deviates from the previous target position, updating the 3D trajectory of the medical instrument to facilitate the medical instrument reaching the target, and
steering the medical instrument toward the target according to the updated 3D trajectory.

44. The method according to claim 43, wherein calculating the planned 3D trajectory from the entry point to the target comprises:
calculating a first planar trajectory for the medical instrument from the entry point to the target, based on a first image or a first set of image frames of a region of interest, the first image frame or first set of image frames pertaining to a first plane, wherein the first planar trajectory is a first 2D trajectory;
calculating a second planar trajectory for the medical instrument from the entry point to the target, based on a second image frame or a second set of image frames of a region of interest, the second image frame or second set of image frames pertaining to a second plane, wherein the second planar trajectory is a second 2D trajectory, and
superpositioning the first and second planar trajectories to form a single 3D trajectory for the medical instrument from the entry point to the target and wherein updating the 3D trajectory comprises:
calculating a 2D trajectory correction on each of two planes; and
superpositioning the two calculated 2D trajectory corrections to form one 3D trajectory correction.

45. The method according to claim 44, wherein the two planes are perpendicular to each other.

46. The method according to claim 44, wherein each of the 2D trajectory corrections is calculated utilizing an inverse kinematics algorithm.

47. The method according to claim 44, further comprising defining at least one of the target and the entry point on the first or second image frames or sets of image frames, using image processing and/or machine learning algorithms.

48. The method according to claim 43, wherein the steering of the medical instrument toward the target within the body is executed utilizing an automated medical device.

49. The method according to claim 43, wherein the real-time position of the medical instrument and/or the target is determined manually by a user.

50. The method according to claim 43, wherein the real-time position of the medical instrument and/or the target is determined automatically by a processor, using image processing and/or machine learning algorithms.

51. The method according to claim 43, further comprising real-time tracking the position of the target within the body, to determine the real-time position of the target within the body.

52. The method according to claim 43, further comprising determining a real-time position of the medical instrument within the body, and tracking the position of the medical instrument within the body to determine the real-time position of the medical instrument within the body.

53. The method according to claim 52, further comprising determining if the real-time position of the medical instrument within the body deviates from the planned 3D trajectory.

54. The method according to claim 53, wherein the determining is performed continuously, wherein the determining is performed at checkpoints along the 3D trajectory and the method further comprises adding and/or repositioning one or more checkpoints along the 3D trajectory.

55. The method according to claim 50, wherein the determining is performed continuously, wherein the determining is performed at the checkpoints along the 3D trajectory and the method further comprises adding and/or repositioning one or more checkpoints along the 3D trajectory.

56. The method according to claim 43, wherein the calculating comprises calculating the planned 3D trajectory such that the medical instrument avoids contact with one or more initial obstacles within the body of the subject.

57. The method according to claim 56, further comprising identifying a real-time location of the one or more initial obstacles and/or one or more new obstacles within the body of the subject and wherein updating the 3D trajectory of the medical instrument comprises updating the 3D trajectory such that the medical instrument avoids entering the real-time location of the one or more initial obstacles and/or the one or more new obstacles.

58. The method according to claim 43, wherein if it is determined that the real-time position of the target deviates from the previous target position, the method further comprises determining if the deviation exceeds a predetermined threshold, and wherein the 3D trajectory of the medical instrument is updated only if it is determined that the deviation exceeds the predetermined threshold.

59. The method according to claim 52, wherein determining the real-time position of the medical instrument within the body of the subject comprises determining the actual position of a tip of the medical instrument within the body of the subject, and wherein determining the actual position of the tip of the medical instrument within the body of the subject comprises:
detecting the medical instrument in one or more images;
defining an end of the detected medical instrument in the one or more images;
determining the position and/or orientation of the medical instrument relative to a coordinate system of an imaging system;
determining a compensation value for the end of the medical instrument based on a look-up table; and
determining the actual position of the tip of the medical instrument in the body of the subject based on the determined compensation value.

60. A system for steering a medical instrument into a target within a body of a subject, the system comprising:
an automated device configured to execute steering of the medical instrument toward the target, the automated device comprising one or more actuators and a control head configured for coupling the medical instrument thereto;
at least one processor configured to:
calculate a planned 3D trajectory for the medical instrument from an entry point to a target in the body of the subject;
generate commands to steer the medical instrument toward the target according to the planned 3D trajectory;
determine if a real-time position of the target deviates from a previous target position;
determine if a real-time position of the medical instrument within the body deviates from the planned 3D trajectory;
update in real-time the 3D trajectory of the medical instrument; and
generate commands to steer the medical instrument toward the target according to the updated 3D trajectory, and
at least one controller configured to control the operation of the automated device based on commands generated by the at least one processor.

61. The system according to claim 60, wherein calculating the planned 3D trajectory from the entry point to the target comprises:
calculating a 2D trajectory from the entry point to the target on each of two planes; and
superpositioning the two calculated 2D trajectories to form a single 3D trajectory, and wherein updating the 3D trajectory comprises:
calculating a 2D trajectory correction on each of two planes; and
superpositioning the two calculated 2D trajectory corrections to form one 3D trajectory correction.

62. The system according to claim 60, wherein the at least one processor is configured to determine if the deviation of real-time position of the target from the previous target position exceeds a set threshold, and wherein the 3D trajectory of the medical instrument is updated only if it is determined that the deviation exceeds the set threshold.

63. A system for updating in real-time a 3D trajectory of a medical instrument, the 3D trajectory extending from an insertion point to a target in the body of a subject, the system comprising:
a processor configured to execute a method for updating in real-time a 3D trajectory of a medical instrument, the 3D trajectory extending from an insertion point to a target in the body of a subject, the method comprising:
defining a real-time position of the target;
defining a real-time position of the medical instrument;
determining if the real-time position of the target deviates from a previous target position and/or if the medical instrument deviates from a planned 3D trajectory based on the defined real-time position of the medical instrument;
if it is determined that the real-time position of the target deviates from the previous target position and/or that the medical instrument deviates from the planned 3D trajectory:
calculating a first planar trajectory correction on a first plane;
calculating a second planar trajectory correction on a second plane; and
determining the 3D trajectory correction for a tip of the medical instrument by superpositioning the first and second planar trajectory corrections.
a monitor configured to display the target, the insertion point and the calculated first and second planar trajectories on one or more image frames; and
a user interface configured to receive input from a user.
